(19) United States
(12) Patent Application Publication
(10) Pub. No.: US 2022/0372417 A1
(43) Pub. Date: Nov. 24, 2022

(54) METHODS AND APPARATUS FOR CELL CULTURE WELLPLATES

(71) Applicant: Agilent Technologies, Inc., Santa Clara, CA (US)

(72) Inventors: Jeff Goldman, Wellesley, MA (US); Jason Dell'Arciprete, Billerica, MA (US)

(21) Appl. No.: 17/773,504

(22) PCT Filed: Oct. 30, 2020
(86) PCT No.: PCT/US20/58223

§ 371 Date: Apr. 29, 2022

Related U.S. Application Data
(60) Provisional application No. 62/928,121, filed on Oct. 30, 2019.

(51) Int. Cl. C12M 1/32; C12M 1/12; C12M 1/00

(52) U.S. Cl. CPC C12M 23/12; C12M 25/06; C12M 23/38

(57) ABSTRACT

The invention relates generally to methods, apparatus and systems for cells in culture. More specifically, the invention relates to novel multiwell plates and concentrator masks. This invention also relates to using the multiwell plates and concentrator masks for cell seeding and for cellular assays.

### CROSS-REFERENCE TO RELATED APPLICATIONS

This application is a US national stage entry of PCT/US20/58223 filed Oct. 30, 2020, which claims the benefit of U.S. Provisional Patent Application No. 62/928,121, filed Oct. 30, 2019, the contents of which are incorporated herein by reference.

### FIELD OF THE INVENTION

The invention relates generally to methods, apparatus and systems for cells in culture. More specifically, the invention relates to novel multiwell plates and concentrator masks. This invention also relates to using the multiwell plates and concentrator masks for cell seeding and for cellular assays.

### BACKGROUND OF THE INVENTION

Multiwell plates are widely used for conducting measurements on cells in parallel and/or simultaneously and are commercially available in a variety of well formats from vendors such as Agilent Technologies, Sigma-Aldrich, Thomas Scientific, and others. Multiwell plates for tissue culture are available in 6, 12, 24, 48, 96, 384- and 1536-well formats, and coated and non-coated plates are available for adherent cell cultures and suspension cultures, respectively.

Multiwell plates are often used for conducting measurements on cell populations. Typically, when cells are seeded into a well of a cell culture well plate, an aqueous cell solution is pipetted into the well, and the cells settle to cover the bottom of the well. The seeded wellplate is often placed into an incubator to promote cellular growth and expansion. This results in cells covering the entire bottom surface of the well. However, there are several challenges that result from having cells seeded on the entire bottom surface of a well, many of which are due to spatial sensitivity associated with many in vitro cellular assays.

Accordingly, it is often desirable to for cultured cells to be concentrated into the center of a well to avoid spatial biases, including but not limited to cell seeding bias die to thermal non-uniformity, related to optical assay sensitivity and edge induced cellular biological effects.

For assays with an optical readout, the efficiency of light transduction from the peripheral area of a well is typically poorer than from the center of the well. This is due to decreased optical access near the well sidewall. Furthermore, illumination and/or data collection efficiency is hindered at the edges of the detector's field of view.

In some instances, plate reader strategies are employed to produce uniform illumination and to facilitate data being captured from the whole well uniformly. However, such strategies are time consuming and inefficient. Other plate reader strategies only make a measurement from the center of the well in order to avoid areas of poor optical transduction, but this approach may omit relevant data for cells that are growing outside of the well center.

Additionally, when cells are seeded into wellplates, cells in the wells on the outside perimeter of the wellplate may grow and behave differently than cells in wells that are not on the perimeter of the plate. This phenomenon is commonly referred to as “edge effect”. This edge effect is significant and, therefore, it is common practice to not seed cells into perimeter wells on a multiwell plate. Without being bound by theory, edge effect is thought to be due to differential heating of the well media in wells on the perimeter of the plate when the plate is placed an in incubator. For example, cells tend to congregate at the well sidewalls in response to the thermal gradient. For wells on the perimeter of the wellplate, the thermal gradient is more severe, causing disproportionate cell proliferation towards the well sidewall. When cells exhibiting an edge effect are analyzed with an optical assay technique, increased well-to-well variability may result. Some well plates contain moats, which the user fills with media, in order to lessen the edge effect. The media is believed to act as a thermal and humidity buffer to the perimeter wells.

There remains a strong need in the art for apparatus, methods and systems for culturing cells in multiwell plates that addresses the physical and biological challenges associated with cells growing close to the side walls of the wells and with optical detection of such cells.

### SUMMARY OF THE INVENTION

These and other features and advantages of the present methods and apparatus will be apparent from the following detailed description, in conjunction with the appended claims.

In one aspect, the present technology is related to a multiwell plate for a cell population in a liquid medium. The multiwell plate comprises: a frame having a frame surface and frame sides extending from the frame surface; a plurality of wells, each well having an open end, a closed end opposite the open end, and at least one wall between the open end and the closed end; wherein the open end of each of the wells is surrounded by the frame surface; wherein the closed end comprises a well surface between and contacting the at least one wall; and at least one continuous ring on the well surface of the closed end of one or more of the wells. It is envisioned the continuous ring can be of any shape as long as the shape comprises a continuous boundary on the closed end of the well. In some embodiments the continuous ring is a circle, oval, or other shape comprising rounded boundary edges. In other embodiments, the continuous ring may be a square, rectangle, triangle or other geometric shape. In certain embodiments, the at least one continuous ring is configured to define at least one cell seeding area on the well surface. The shape of the cell seeding area is defined by the shape of the continuous ring and is envisioned to be of any shape so long as it comprises a continuous boundary. In some embodiments, the multiwell plate comprises more than one continuous ring configured to define more than one cell seeding area.

In another aspect, the present technology is related to a concentrator mask for seeding cells in liquid medium into a multiwell plate. The concentrator mask comprises: a frame having a frame surface and frame sides extending from the frame surface; a plurality of funnels extending from the frame surface, each funnel having a first open end and a second open end; wherein the first open end is connected to the frame surface and has a larger diameter than the second open end.

Other aspects of the present technology are related to methods of seeding cells in a central portion of a culture well. The method comprises: pipetting a liquid medium comprising cells into the at least one cell seeding area that is circumscribed by at least one continuous ring on the surface of the closed end of each well.

Other aspects of the present technology are related to a cell seeding system comprising a multiwell plate and a concentrator mask. The multiwell plate comprises a frame having a frame surface and frame sides extending from the frame surface; a plurality of wells, each well having an open end, a closed end opposite the open end, and at least one wall between the open end and the closed end. The open end of each of the wells is surrounded by the frame surface; wherein the closed end comprises a well surface between and contacting the wall; and at least one continuous ring on the well surface of the closed end of one or more of the wells. The concentrator mask comprises a frame having a frame surface and frame sides extending from the frame surface; and a plurality of funnels extending from the frame surface. Each funnel has a first open end and a second open end, and the first open end is connected to the frame surface and has a larger diameter than the second open end. The plurality of funnels of the concentrator mask and the multiwell plate form the cell seeding system when the funnels of the concentrator mask are inserted into the plurality of wells so that the second open ends of the funnels make a contact with the continuous rings on the well surface of the closed end of each well.

Other aspects of the present technology are related to methods of seeding cells in the center of a well on a multiwell plate by pipetting a liquid medium comprising cells into the first open end of a funnel of a cell seeding system, and the cells are deposited into an area circumscribed by the at least one continuous ring on the well surface.

### BRIEF DESCRIPTION OF THE DRAWINGS

FIG. 1A is a cut away view showing a cross-section of a multiwell plate having wells comprising a continuous ring on the inner bottom surface of the well.

FIG. 1B is an enlarged in view of FIG. 1A showing a multiwell plate having wells comprising a continuous ring on the inner bottom surface of the well.

FIG. 2A is a schematic illustration of a concentrator mask for seeding cells into a multiwell plate.

FIG. 2B is a cut away view of FIG. 2A showing a cross-section of a concentrator mask for seeding cells into a multiwell plate.

FIG. 3 is a cut away view of a cell seeding system comprising a concentrator mask inserted into a multiwell plate having wells comprising a continuous ring on the inner bottom surface of the well.

FIG. 4 is a schematic illustration of a 96 well plate having wells comprising a continuous ring on the inner bottom surface of the well.

The present teachings are best understood from the following detailed description when read with the accompanying drawing figures. The features are not necessarily drawn to scale. Wherever practical, like reference numerals refer to like features.

### DETAILED DESCRIPTION OF THE INVENTION

It is to be understood that the terminology used herein is for purposes of describing particular embodiments only, and is not intended to be limiting. The defined terms are in addition to the technical and scientific meanings of the defined terms as commonly understood and accepted in the technical field of the present teachings.

### Definitions

As used herein, and in addition to their ordinary meanings, the terms “substantial” or “substantially” mean to within acceptable limits or degree to one having ordinary skill in the art.

As used herein, the terms “approximately” and “about” mean to within an acceptable limit or amount to one having ordinary skill in the art. The term “about” generally refers to plus or minus 15% of the indicated number. For example, “about 10” may indicate a range of 8.5 to 11.5. For example, “approximately the same” means that one of ordinary skill in the art considers the items being compared to be the same. In the present disclosure, numeric ranges are inclusive of the numbers defining the range.

Before the various embodiments are described, it is to be understood that the teachings of this disclosure are not limited to the particular embodiments described, and as such can, of course, vary. It is also to be understood that the terminology used herein is for the purpose of describing particular embodiments only, and is not intended to be limiting, since the scope of the present teachings will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are now described. All patents and publications referred to herein are expressly incorporated by reference.

As used in the specification and appended claims, the terms “a,” “an,” and “the” include both singular and plural referents, unless the context clearly dictates otherwise. Thus, for example, “a well” includes one well and plural wells.

Multiwell Plate with Wells Having a Continuous Ring

In at least one embodiment, the current technology is related to a continuous ring molded into the bottom of each well in a cell culture or assay wellplate. The continuous ring creates a central cell seeding area on the well bottom. When cells are seeded into the center of the ring, the ring acts as a physical barrier to constrain cells to the center of the well. This has several advantages outlined below.

In addition, when combined with certain microwell assay instruments, such as the Agilent XF instruments, the continuous ring acts to define an assay microchamber volume which enables increased assay sensitivity. For example, in some embodiments, the continuous ring of the wells interfaces with analysis instruments to form a semi-sealed, transient, reduced-volume microchamber where metabolic measurements can be made.

In some embodiments of the present technology, cells are seeded into the center of the well, within the boundary formed by the continuous ring. The continuous ring provides a physical barrier that constrains seeded cells to the center of the well. Seeding cells only in the center of the well, away from the well sidewall, provides mitigation of the effects caused by optical transduction and cell seeding density gradients due to temperature differences and/or fluidic differences near the perimeter of a well. Because the cells are distant from the well sidewalls, the impact of illumination differences are also minimized.

In other embodiments, the present technology is related to methods for seeding cells inside the continuous ring on the well bottom of a multiwell plate. In some embodiments, cells in a liquid medium are added (e.g., pipetted) into the cell seeding area defined by the continuous ring. In these embodiments, the cells can then be expanded, for example by incubation, but the cell expansion is restricted to the cell seeding area formed by the continuous ring. The presently described multiwell plates, and methods of using the same, are compatible with both adherent and suspension cells. In some other embodiments, cells, tissues, or organoids can be added (e.g., seeded) into particular regions formed by the continuous ring(s).

In other embodiments, cells can be seeded both inside and outside of the continuous ring, such as to create a non-contact co-culture of different cell types. In some embodiments, the cell seeding areas comprise cell culture media that is restricted to a cell seeding area by the continuous ring/rings. In other embodiments, the cell seeding areas comprise cell culture media that is in fluid communication with the cell culture media from other cell seeding areas.

In some embodiments, the wells of the multiwell plates of the present technology may be coated with a substance that promotes cell adherence in order to facilitate adherence of suspension cells, or improve adherence of adherent cells, to the well surface. For example, polycationic coatings, e.g. poly-1-lysine, may be used to promote adherence of any cell type to the surface of the well. In other embodiments, uncoated multiwell plates may be used.

In additional embodiments, the present technology is related to preparation and maintenance of non-contact cell co-culture of different cells types either inside or outside of the continuous ring. It is also envisioned that the continuous ring for providing non-contact co-culture can be of any shape as long as the shape comprises a continuous boundary on the closed end of the well. In some embodiments the continuous ring for non-contact co-culture is a circle, oval, or other shape comprising rounded boundary edges. In other embodiments, the continuous ring for non-contact co-culture may be a square, rectangle, triangle or other geometric shape.

In certain embodiments, at least one continuous ring, of any shape, is configured to define at least one cell seeding area on the well surface. In other embodiments, multiple sets of continuous rings, such as, but not limited to, concentric rings, are configured to define more than one cell seeding area on the well surface. In other embodiments, the molded physical barrier on the well surface may not be a ring but instead be configured to provide a grid or other configuration of cell seeding areas.

When more than one cell seeding area is present on the well surface, it is envisioned that each cell seeding area may contain a different cell type, the same cell type, a mixture of cell types, or any combination of the forgoing as desired by the skilled artisan. In any event, the shape and number of the cell seeding areas are defined by the shape and number of the continuous rings and are envisioned to be of any shape, size and/or configuration to provide a continuous boundary and/or boundaries to the cell seeding area and/or areas.

Additional embodiments of the present technology are related to using the microwell plates in conjunction with an optical fiber probe, such as probes used in the Agilent XF instruments. In these embodiments, seeding cells only in the center of the well minimizes differences in optical signal caused by radially differential optical signal transduction. This results in both an increase in measurement sensitivity and a more uniformly represented signal transduction for the entire well.

Without being bound by theory, it is contemplated that cells constrained to the center of the wells are less impacted by thermal gradients within the well that occur during the cell culture workflow and assay. Additionally, since the cells are constrained to the center of the well, they remain in a position where the optical transduction is highest, allowing for increased detection sensitivity and reduced assay variability that would be introduced by cells seeded at the perimeter of the wells.

In other embodiments, the wells comprising continuous rings form smaller assay microchambers that enable greater assay sensitivity using microwell plate readers, such as, Agilent XF instruments or other well based measurement assays that have a radial dependency on signal transduction or a radial dependency on assay cell seeding area or assay volume. Without being bound by theory, it is also contemplated that the cells seeded in the cell seeding areas formed by the continuous rings will exhibit reduced edge effects allowing all wells in a wellplate to be used in assays with improved well to well analysis and uniformity.

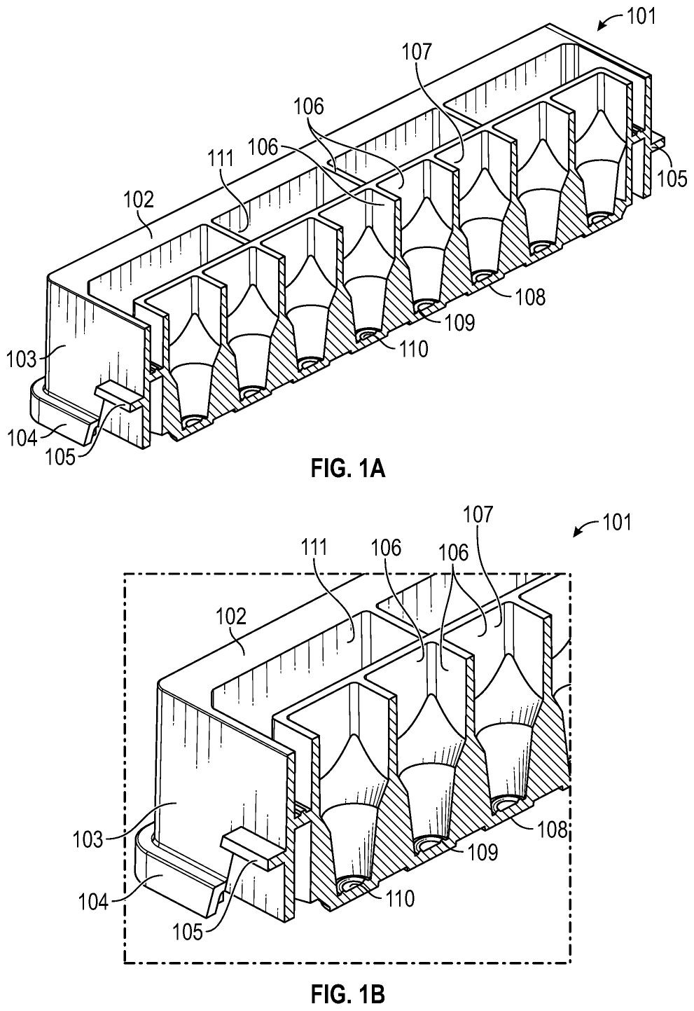

FIG. 1A is a cut away view showing a cross-section of an embodiment of the present multiwell plate 101 having wells comprising a continuous ring on the inner bottom surface of the well. In this embodiment, the multiwell plate 101 is defined by a frame having a frame surface 102, a frame side 103 and a frame base 104. The frame of this embodiment also includes a frame tab 105 for multiwell plate handling. Multiwell plate 101 also comprises well walls 106, open ends 107 and closed ends 108. The closed ends of the wells 108 further comprise continuous rings 109 that define cell seeding areas 110. In this embodiment, the multiwell plate 101 also comprises perimeter well plate moats 111, however in other embodiments, the multiwell plates of the present technology do not comprise perimeter well plate moats. FIG. 1B is an enlarged view of FIG. 1A, showing an embodiment of the present multiwell plate having wells comprising a continuous ring on the inner bottom surface of the well.

In some embodiments, the cell seeding area that is encircled by the continuous ring has an area of about 10% to about 80% of a total area of the well surface. In other embodiments, the cell seeding area that is encircled by the continuous ring has an area of about 55% to about 75% of a total area of the well surface. In some embodiments, the cell seeding area that is encircled by the continuous ring has an area of about 60% to about 70% of a total area of the well surface. In other embodiments, the cell seeding area is about 65% of a total area of the well surface. In other embodiments the cell seeding area is about 10% to about 25% of a total area of the well surface.

In some embodiments, the continuous ring has a height of about 0.01 mm to about 2 mm. In other embodiments, the continuous ring has a height of about 0.1 mm to about 0.5 mm or about 1 mm. In other embodiments, the continuous ring has a height of about 0.3 mm to about 0.8 mm. In some embodiments, the continuous ring has a height of about 0.2 mm tall.

In some embodiments, the continuous ring has an inner diameter of about 0.5 mm to about 6.0 mm. In other embodiments, the continuous ring has an inner diameter of about 1.0 mm to about 5.0 mm. In some embodiments, the continuous ring has an inner diameter of about 3.0 mm to about 4.0 mm. In some embodiments, the continuous ring has an inner diameter of about 2.0 mm.

The multiwell plates of the present technology can be configured in any manner or orientation including to have dimensions that are consistent with the well number and spacing of standard multiwell plates. For example, in some embodiments, a multiwell plate of the present technology comprises at least 8 wells. In other embodiments, a multiwell plate of the present technology comprises at least 24 wells. In some embodiments, a multiwell plate of the present technology comprises at least 48 wells. In other embodiments, a multiwell plate of the present technology comprises at least 96 wells. In other embodiments, a multiwell plate of the present technology comprises at least 384 wells. In other embodiments, a multiwell plate of the present technology comprises at least 1536 wells.

### Concentrator Mask for Cell Seeding

As another aspect, the present technology provides a cell seeding concentrator mask comprising a plurality of funnels. In some embodiments, the plurality of funnels is configured so that the funnel shaped well inserts can be inserted into a strip or grid of wells in a wellplate. In some embodiments, the cell seeding concentrator mask is used to constrain cell seeding into an area within a well that is smaller than the well bottom. In some embodiments, the concentrator mask comprises a frame and a plurality of funnels extending from the frame. In other embodiments, the funnels of the concentrator mask comprise a first open end and a second open end wherein the first open end is connected to the frame and has a larger diameter than the second open end of the funnel.

The cell seeding concentrator masks of the present technology enable cells to be seeded into a selected areas within a larger well using standard pipets and techniques. In some embodiments, the cell seeding concentrator masks restrict the seeded cells to the central well area during incubation. The concentrator masks of the present technology can be used for seeding adherent or suspension cells.

In some embodiments, the wells of the multiwell plates may be coated with a substance that promotes cell adherence in order to facilitate adherence of suspension cells or improve adherence of adherent cells to the well surface. For example, polycationic coatings, e.g. poly-1-lysine, may be used to promote adherence of any cell type to the surface of the well. In other embodiments, uncoated multiwell plates may be used.

In some embodiments, the second open ends of the plurality of funnels are configured to be smaller than the bottom of the well into which the funnel is inserted. In these embodiments, the concentrator mask can be used for seeding cells in the center of the well and not on the edges of the well close to the well perimeter wall.

In certain embodiments, the present technology is related to methods whereby cells in a liquid solution are pipetted into the first open end of a concentrator mask funnel that is inserted into a well of a multiwell plate and the cells are allowed to settle. In other embodiments, the multiwell plate is spun in a centrifuge such that the cells are spun onto an adherent coating on the well bottom. The multiwell plate can then be moved to an incubator to promote cell growth and expansion. In some embodiments, the concentrator mask remains in the wellplate during incubation. In other embodiments, the concentrator mask can be removed from the well plate prior to incubation. In additional embodiments, the concentrator mask remains in the wellplate during incubation and is removed prior to analysis of the seeded cells.

In another embodiment of the present technology, the distal end of the second open end of the funnel creates an interface with the well bottom. In some embodiments, the interface is a liquid-tight seal but in other embodiments the interface allows but reduces liquid passage, or diffusion, such as by providing a gap. In this embodiment, a cell solution is pipetted into the first open end of a funnel and the solution fills the well both inside and outside of the second open end of the funnel. Without being bound by theory, it is contemplated that cells settle to the well bottom by gravity, and therefore, the number of cells which deposit on an area of the well bottom is dependent on the number of cells in suspension above it. Accordingly, the cell concentrator mask of this embodiment is designed such that it physically occupies a volume above the areas of the well bottom where cells are not desired. Thus, the final result of seeding cells in accordance with this embodiment of the technology is cells seeded at high concentration in the center of the well while no or low concentration of cells are seeded at the peripheral area of the well close to the well wall.

In certain embodiments, the outer diameter of the second open end of the funnel is configured to substantially match the inner diameter of the well in which it is inserted, so that the inserted funnel is held in place by compression or an interference fix. In some embodiments, the outer diameter is configured to provide a predetermined gap with the inner diameter of the well or a portion thereof. The funnels of the present technology can be configured for insertion into wells of any size. Additionally for this embodiment, the size of the area in which high concentration of cells are seeded is determined by the inner diameter of the second open end of the funnel and how the funnel is configured to be inserted into the wells of the multiwell plate.

In an additional embodiment, the present technology provides a cell concentrator mask comprising a funnel having a second open end, wherein the second open end comprises a distal elastomer portion. In some embodiments, the distal elastomer portion of the second open end creates an interface with the well bottom that is a liquid proof seal. In this embodiment, a cell suspension can be pipetted into the first open end of a funnel and into the bottom of the well, displacing air from the funnel bottom with the liquid cell solution. In this embodiment, the cell suspension will only seed cells at the bottom of the well inside the inner diameter of the second open end of the funnel. In this embodiment, seeded cells settle to the well bottom with the funnel inserted into a well. The multiwell plate can then be moved to an incubator to promote cell growth and proliferation. In some embodiments, the concentrator mask remains in the wellplate during incubation. In other embodiments, the concentrator mask can be removed from the well plate prior to incubation. In additional embodiments, the concentrator mask remains in the wellplate during incubation and is removed prior to analysis of the seeded cells. In particular embodiments of the present technology, an area of concentrated cells is seeded in the center of a well while cells are absent from the well perimeter near the well wall edge.

In some embodiments, the distal elastomer portion of the second open end comprises a compliant seal material, such as a resilient, essentially fluid impermeable material in the form of an o-ring. The compliant seal material can be any shape suitable for the end of the second open end. For example, the compliant seal material may be a toroidal-shaped o-ring, a gasket with a rectangular cross-section, a metallic gasket, or another type of compliant material. In one embodiment, the compliant seal material can be a fluoroelastomer material or other material which will form a fluidic seal with an opposing well surface. In another embodiment, the compliant seal material is silicone rubber. In some embodiments, the compliant seal material makes a radial seal between the second open end and the well surface. It is also contemplated that other seal orientations may be employed. The compliant seal material can be various rubbers depending on the temperatures used and the other cell culture media components and conditions, e.g. fluoropolymers, buna-n, EPDM or, in some cases, metallic with compliant over-plating. The compliant seal material may also be coated with a chemically inert, biologically compatible coating if the material of the o-ring allows for it.

In some embodiments, the concentrator masks of the present technology are configured to interface with a flat bottom cell culture well. In other embodiments, the concentrator masks of the present technology are configured to interface with a dimpled well, such as, for example, an Agilent XF well.

In yet other embodiments, the concentrator masks of the present technology are configured to interface with a well of the present technology that comprises a continuous ring molded into the bottom of the well.

Accordingly, the concentrator masks of the present technology can be used in methods that seed cells in a central cell seeding area within a larger well. In some embodiments, the concentrator mask funnels are used in methods that concentrate cell seeding at the center of a well bottom. In some embodiments, the concentrator masks seed the majority of the cells above a central well bottom area at a desired cell concentration. In other embodiments, the concentrator masks of the present technology can be used in a method that excludes cell seeding from areas of a well bottom where cell are not desired.

In some embodiments, the cell seeding concentrator masks of the present technology can be used with cell suspensions in conjunction with centrifugation and surface coatings so that the cells in suspension are adhered to the well surface. In certain embodiments, the concentrator masks can be removed prior to downstream analysis.

The present methods can further comprise analysis of the cells, such as analysis by Agilent XF assays.

In some methods of the present technology, the methods further comprise removing air that is trapped at the concentrator mask/well bottom interface, such as by pipetting action.

In some embodiments, about 5.0 μl to about 20 μl of media can be pipetted into a concentrator mask. In other embodiments, about 10 μl to about 15 μl of media can be pipetted into a concentrator mask. In some embodiments, about 12.5 μl of media can be pipetted into a concentrator mask. Accordingly, the methods of the current technology can be performed using volumes that do not introduce significant pipetting error.

In some embodiments of the present technology, the seeded multiwell plates can be moved to an incubator for cell expansion. In some of these embodiments, the concentrator mask can remain in place during cell incubation. Accordingly, certain embodiments of the present technology are compatible with low adherence cells. Other embodiments of the present technology are related to culture of high adherence cells.

In other embodiments, the concentrator masks and methods of using the same are compatible with commercially available multiwell plates, such as Agilent XF wellplates. In additional embodiments, the concentrator masks and methods of using the same are compatible with multiwell plates of the current technology that comprise a continuous ring on the inner bottom well surface.

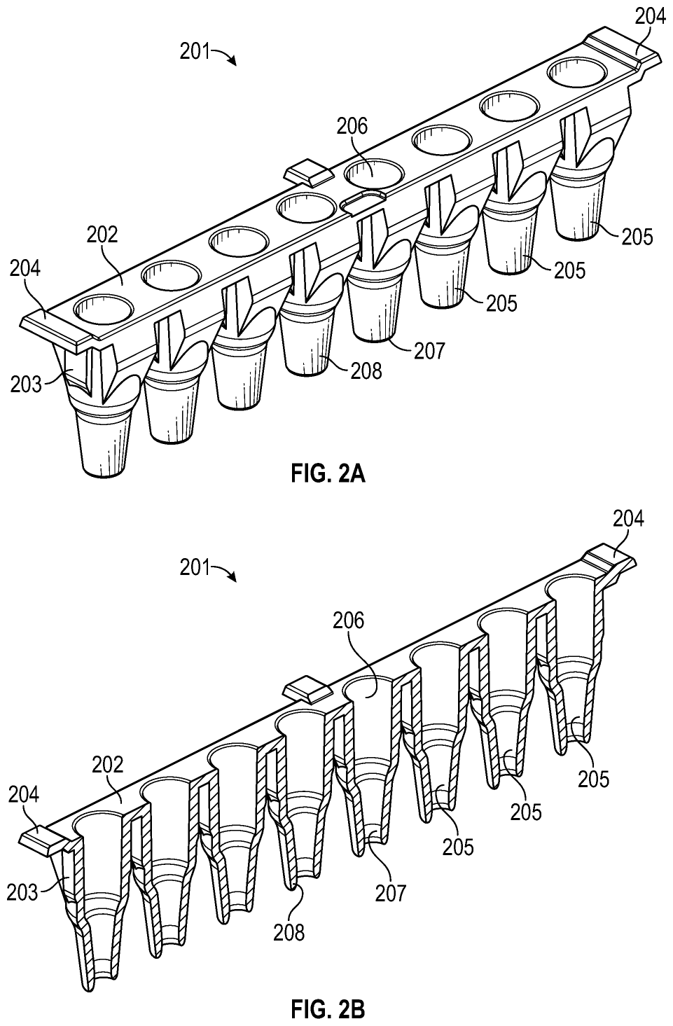

FIG. 2A is a schematic illustration of a concentrator mask for seeding cells into a multiwell plate. In this embodiment, concentrator mask 201 is defined by frame surface 202 and frame sides 203. In this embodiment, the frame also comprises frame tabs 204 for alignment and stacking of the concentrator masks onto multiwell plates. A plurality of funnels 205 extend from the frame. The funnels comprise a first open end 206 and a second open end 207 wherein the first open end 206 has a larger diameter than the second open end 207. In some embodiments, the distal end 208 of the funnel comprises an elastomer portion. In some embodiments, the funnel 205 is inserted into a well wherein a liquid proof interface is created between the bottom of the well and the distal end 208 of the funnel. FIG. 2B is a cut away schematic illustration of FIG. 2A showing a cross-section of a concentrator mask for seeding cells into a multiwell plate.

The concentrator masks of the present technology can be configured in any manner or orientation, including to have the appropriate number and spacing of funnels to be compatible with and fit into standardized multiwell plates. For example, in some embodiments, the concentrator mask of the present technology comprises at least 8 funnels. In other embodiments, the concentrator mask of the present technology comprises at least 12 funnels. In other embodiments, the concentrator mask of the present technology comprises at least 24 funnels. In some embodiments, the concentrator mask of the present technology comprises at least 48 funnels. In other embodiments, the concentrator mask of the present technology comprises at least 96 funnels. In other embodiments, the concentrator mask of the present technology comprises at least 384 funnels. In other embodiments, the concentrator mask of the present technology comprises at least 1536 funnels.

In some embodiments, the concentrator masks of the present technology are configured to comprise funnels having an inner diameter of about 0.5 mm to about 6.0 mm. In some other embodiments, the concentrator masks of the present technology are configured to comprise funnels having an inner diameter of about 1.0 mm to about 5.0 mm. In other embodiments, the concentrator masks of the present technology are configured to comprise funnels having an inner diameter of about 3.0 mm to about 4.0 mm. In other embodiments, the concentrator masks of the present technology are configured to comprise funnels having an inner diameter of about 2.0 mm.

### Cell Seeding System Comprising Multiwell Plate and Concentrator Mask

The present technology is also related to a cell seeding system comprising a concentrator mask as described herein that is used in conjunction with a continuous ring multiwell plate, also as described herein. In this embodiment, the plurality of funnels of a concentrator mask are configured to fit into a plurality of wells of a multiwell plate.

The cell seeding system of the present technology includes a multiwell plate comprising a frame having a frame surface and frame sides extending from the frame surface; a plurality of wells, each well having an open end, a closed end opposite the open end, and at least one wall between the open end and the closed end; wherein the open end of each of the wells is surrounded by the frame surface; wherein the closed end comprises a well surface between and contacting the wall; and at least one continuous ring on the well surface of the closed end of each well.

The cell seeding system also comprises a concentrator mask comprising a frame having a frame surface and frame sides extending from the frame surface; a plurality of funnels extending from the frame surface, each funnel having a first open end and a second open end; and wherein the first open end is connected to the frame surface and has a larger diameter than the second open end.

In the cell seeding systems of the present technology, the plurality of funnels of the concentrator mask is configured to fit into the plurality of wells of the multiwell plate so that the second open ends of the funnels make a contact with the continuous rings on the well surface of the closed end of each well. In some embodiments, a bottom portion of the distal end contacts a top portion of the continuous ring. In some embodiments, a lateral portion of the distal end contacts a lateral portion of the continuous ring.

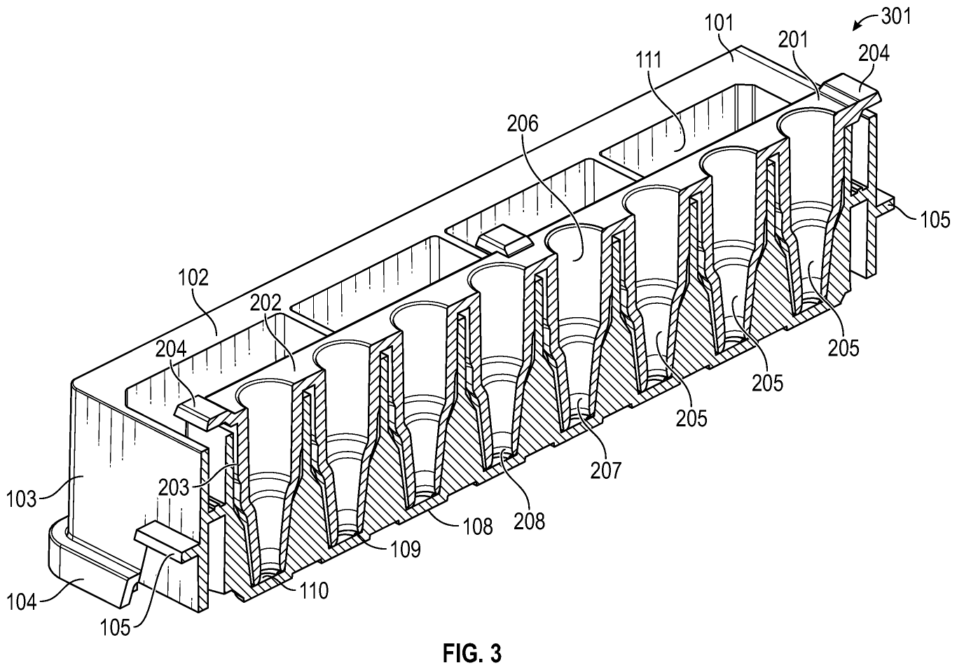

FIG. 3 is a cut away schematic illustration of a cell seeding system comprising a concentrator mask inserted into a multiwell plate having wells comprising a continuous ring on the well surface of the closed end of the well. In this embodiment, the cell seeding system 301 is formed by a concentrator mask 201 and a multiwell plate 101 that are configured to fit together so that the funnels of the concentrator mask 201 are inserted into the wells of the multiwell plate 101. The cell seeding system 301 comprises a multiwell frame surface 102, multiwell frame sides 103 and a multiwell frame base 104. In this embodiment, the multiwell frame also comprises multiwell frame tabs 105 for plate handling. The multiwell plate comprises well walls 106 (not visible) that define the wells, and, in this embodiment, a perimeter well plate moat 111. The multiwell plate wells also comprise open ends (not visible) and closed ends 108 that include continuous rings 109. The continuous rings 109 form cell seeding areas 110 on the central portion of closed well end 108.

The concentrator mask 201 of the cell seeding system 301 comprises a frame surface 202 and frame sides 203. The concentrator mask also comprises a plurality of funnels 205. The funnels comprise a first open end 206 and a second open end 207. The concentrator mask 201 is configured so that the second open ends 207 of the funnels are inserted into the wells of the multiwell plate 101. In this embodiment, the second open ends 207 of the funnel are configured to contact the continuous rings 109 on the well surface of the closed end of the wells 108. In some embodiments, the second open ends 207 comprise a distal portion 208. In some embodiments, the interface between the second open end 207 and the continuous ring 109 creates a liquid-tight seal. In certain embodiments, the interface between the second open end 207 and the continuous ring 109 does not form a liquid-tight seal. The cell seeding system also comprises multiwell plate frame tabs 105 for plate handling. The cell seeding system also comprises concentrator mask frame tabs 204 for alignment and insertion of the concentrator mask 201 into the multiwell plate 101.

The present technology is also related to methods of using the cell seeding system 301 for seeding cells into the center of a wells in a multiwell plate. In some embodiments, the method comprises adding a liquid medium comprising cells into the first open funnel end 206 of the cell seeding system so that the cells are deposited into a cell seeding area 110 defined by at least one continuous ring 109 on the well surface of the closed end of a well 108. In some embodiments, the seeded cell seeding system 301 can be moved to an incubator for cell expansion. In certain embodiments, the concentrator mask 201 can be removed from the multiwell plate 101. In other embodiments, the concentrator mask 201 can remain inserted into the multiwell plate 201 of the cell seeding system 301. In some embodiments, the concentrator mask 201 is removed from the multiwell plate 101 prior to incubation. In other embodiments, the concentrator mask 201 can remain inserted into the multiwell plate 101 while the cells are incubated for expansion. In some embodiments, the concentrator mask 201 can be removed from the multiwell plate 101 prior to analysis of the cells. In other embodiments, the concentrator mask 201 can remain in the multiwell plate 101 during analysis of the cells. In some embodiments, the analysis of the cells includes an optical readout.

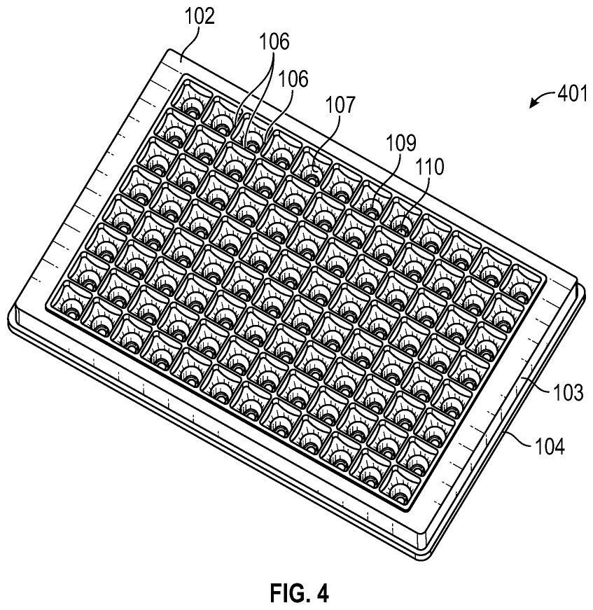

The present technology is related to and compatible with 6, 12, 24, 48, 96, 384- and 1536-well formats. FIG. 4 is a schematic illustration of a 96 well plate, of the present technology, having wells comprising a continuous ring on the inner bottom surface of the well. In this embodiment, the multiwell plate 401 is defined by a frame having a frame surface 102, a frame side 103 and a frame base 104. Multiwell plate 401 also comprises well walls 106, open ends 107 and closed ends 108 (not visible). The closed ends of the wells 108 further comprise continuous rings 109 that define cell seeding areas 110. In this embodiment, the multiwell plate 401 does not include perimeter well plate moats or a frame tab for plate handling, however in other embodiments, the multiwell plates of the present technology include perimeter well plate moats and frame tabs for handling.

### Metabolic Measurements

In some embodiments, the present methods, apparatus and systems are useful in measuring cell biology, such as in the area of micro-respirometry, which includes quantitatively measuring the bioenergetics or metabolic state of a small number of cells, as opposed to respirometry performed on whole animals. In the past, micro-respirometry was performed with microscopic glass flow cells that utilized milliliters of cell culture and Clark electrodes for measuring cell metabolism. This technique is not microscopic, facile or high-throughput. Flux analyzers and assays from Seahorse Bioscience provided improved technology for micro-respirometry by introducing comprehensive assays that can be easily performed in 8, 24 and 96 plastic cell culture plates. The resulting technology enabled complex characterization of both the glycolysis and oxidative phosphorylation pathways, by introducing various stimulants, inhibitors and custom drugs and measuring changes in oxygen consumption and proton production. Additional details related to micro-respirometry are provided in U.S. patent application Ser. No. 15/896,255, which is hereby incorporated by reference in its entirety.

As another aspect of the present invention, the present methods, apparatus and systems are used for metabolic measurements of individual cell types in a culture. In some embodiments, the present methods, apparatus and systems can be used for analysis of cells in a non-contact co-culture. The system can include a multiwell plate as described herein. For example, the multiwell plate described herein can be used to maintain a non-contact co-culture by placing a first cell type inside the cell seeding area circumscribed by the continuous ring and a second cell type outside the continuous ring, between the continuous ring and the well wall. In some embodiments, the different cell types in the non-contact co-culture are in fluid communication with each other. In other embodiments, the different cells types of the non-contact co-culture are not in fluid communication with each other.

Additional aspects of the present invention include more than one continuous ring, of any shape, wherein the continuous rings provide multiple cell seeding areas or segments of any size, shape or configuration for preparation and maintenance of a non-contact co-culture by seeding different cells into different cell seeding areas/segments. In addition, certain aspects of the present technology comprise a molded physical barrier on the well surface that is not a ring but is configured to provide a grid or alternative configurations of cell seeding areas. When a non-contact co-culture of the present technology is produced by seeding different cell types into different cell seeding areas, the present methods, apparatus and systems may be used to independently obtain metabolic measurements from one cell seeding area/segment at a given time. Alternatively, the present methods apparatus and systems may be used to obtain metabolic measurements from two or more, or all of the, cell seeding areas/segments at a given time. Additional details related to non-contact co-culture are provided in U.S. patent application Ser. No. 15/896,255, which is hereby incorporated by reference in its entirety.

In the present methods, apparatus and systems, one or more sensors can be used to measure physiological properties of a cell population. The sensors can be a fluorescent sensor, a luminescent sensor, an ISFET sensor, a surface plasmon resonance sensor, a sensor based on an optical diffraction principle, a sensor based on a principle of Wood's anomaly, an acoustic sensor, or a microwave sensor. The present technology is not limited to any particular cellular assays, measurements or sensors but, instead, can be used by the skilled artisan in conjunction with any desired cellular analysis approaches. Accordingly, the present systems, apparatus and methods can comprise one or more of the foregoing sensors positioned to measure one or more properties of a sample within the wells described herein.

The present methods, apparatus and systems can be used in a variety of fields related to cell culture and analysis. Such fields include, but are not limited to, biological research, drug discovery, and clinical diagnostics. For example, as a drug discovery tool, the device can be used to screen various molecules for an effect on cellular metabolism in co-culture, protein secretion, or intra/extra cellular ion exchange. The present methods, apparatus and systems can also be used to determine the health of cells in culture, including co-culture, both before and after a conventional assay is performed, thereby improving the performance of such an assay.

### Cell Populations

The cell populations used in the present methods and apparatus may include any cells of interest. Such cells include, but are not limited to, bacteria, fungus, yeast, a prokaryotic cell, a eukaryotic cell, an animal cell, a human cell, and/or an immortal cell. At least a portion of the cells may be attached to a surface of the vessel. At least a portion of the cells may be suspended in the media. At least a portion of the cells may include living tissue, organoids, spheroids or engineered tissue. In some embodiments, at least a portion of cells are adhered to a closed end or a wall of the wells.

Known cell lines can be used as a cell type in the present methods, apparatus, and systems. For example, known cell lines that can be used in conjunction with the present technology include, but are not limited to, C8161, CCRF-CEM, MOLT, mIMCD-3, NHDF, HeLa, HeLa-S3, Huh1, Huh4, Huh7, HUVEC, HASMC, HEKn, HEKa, MiaPaCell, Panc1, PC-3, TF1, CTLL-2, CIR, Rath, CV1, RPTE, A10, T24, J82, A375, ARH-77, Calu1, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRC5, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9 L, A2780, A2780ADR, A2780cis, A172, A20, A253, A431, A-549, ALC, B16, B35, BCP-1 cells, BEAS-2B, bEnd.3, BHK-21, BR 293, BxPC3, C3H-10T1/2, C6/36, Cal-27, CHO, CHO-7, CHO-IR, CHO-K1, CHO-K2, CHO-T, CHO Dhfr−/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepa1c1c7, HL-60, HMEC, HT-29, Jurkat, JY cells, K562 cells, Ku812, KCL22, KG1, KYO1, LNCap, Ma-Me1 1-48, MC-38, MCF-7, MCF-10A, MDA-MB-231, MDA-MB-468, MDA-MB-435, MDCK II, MDCK II, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof. Cell lines are available from a variety of sources known to those with skill in the art (see, e.g., the American Type Culture Collection (ATCC) (Manassas, Va.)). These or other cell lines can be employed as a first cell type or a second cell type in the present methods and apparatus. In some embodiments, the first cell type is in a cell population taken from a subject (such as a human patient), and the second cell type is a known cell line.

### EXEMPLARY EMBODIMENTS

1. A multiwell plate for a cell population in a liquid medium, the multiwell plate comprising:

a frame having a frame surface and frame sides extending from the frame surface;

a plurality of wells, each well having an open end, a closed end opposite the open end, and at least one wall between the open end and the closed end; wherein the open end of each of the wells is surrounded by the frame surface; wherein the closed end comprises a well surface between and contacting the at least one wall; and

at least one continuous ring on the well surface of the closed end of one or more of the wells.

2. The multiwell plate of embodiment 1, wherein the at least one continuous ring is configured to define at least one cell seeding area on the well surface, which can form an assay microchamber in cooperation with a lid, a plunger, or another element.

3. The multiwell plate of embodiment 1, wherein the multiwell plate comprises more than one continuous ring configured to define more than one cell seeding area.

4. The multiwell plate of any of the foregoing embodiments, wherein the at least one continuous ring has a height of about 0.01 mm to about 2 mm.

5. The multiwell plate of any of the foregoing embodiments, wherein the at least one continuous ring has an inner diameter of about 0.5 mm to about 6.0 mm, for example, an inner diameter of about 2.0 mm.

6. A concentrator mask for seeding cells in liquid medium into a multiwell plate, the concentrator mask comprising:

a frame having a frame surface and frame sides extending from the frame surface;

a plurality of funnels extending from the frame surface, each funnel having a first open end and a second open end;

wherein the first open end is connected to the frame surface and has a larger diameter than the second open end.

7. The concentrator mask of embodiment 6, wherein the second open end comprises a distal elastomer portion.

8. The concentrator mask of embodiment 6 or 7, wherein the concentrator mask comprises at least 8 funnels.

9. The concentrator mask of embodiment 6 to 8, wherein the funnels have an inner diameter of about 0.5 mm to about 6.0 mm, for example, an inner diameter of about 2.0 mm.

10. A method of seeding cells in a central portion of a culture well, the method comprising:

adding a liquid medium comprising cells into at least one cell seeding area on the well surface of any of embodiments 1 to 5.

11. The method of embodiment 10, wherein the culture well is moved to an incubator and incubated after seeding with cells.

12. The method of embodiment 11, wherein the incubated cells are substantially free of or do not exhibit any incubator induced edge effects.

13. A cell seeding system comprising a multiwell plate and a concentrator mask:

wherein the multiwell plate comprises a frame having a frame surface and frame sides extending from the frame surface; a plurality of wells, each well having an open end, a closed end opposite the open end, and at least one wall between the open end and the closed end; wherein the open end of each of the wells is surrounded by the frame surface; wherein the closed end comprises a well surface between and contacting the wall; and at least one continuous ring on the well surface of the closed end of each well;

wherein the concentrator mask comprises a frame having a frame surface and frame sides extending from the frame surface; a plurality of funnels extending from the frame surface, each funnel having a first open end and a second open end; and wherein the first open end is connected to the frame surface and has a larger diameter than the second open end; and

wherein the plurality of funnels is configured to fit into the plurality of wells so that the second open ends of the funnels form an interface with the continuous rings on the well surface of the closed end of one or more of the wells.

14. The system of embodiment 13, wherein the interface between the second open end and the continuous ring is a gap sufficiently small to reduce liquid diffusion.

15. The system of embodiment 13, wherein the second open end comprises a distal elastomer portion.

16. The system of embodiment 15, wherein the interface is physical contact between the distal elastomer portion of the second open end and the continuous ring, thereby forming a liquid-tight seal.

17. A method of seeding cells in a multiwell plate, the method comprising;

adding a liquid medium comprising cells into the first open end of the cell seeding system of embodiment 13, wherein the cells are deposited into an area circumscribed by the at least one continuous ring on the well surface.

18. The method of embodiment 17, wherein the multiwell plate is moved to an incubator after seeding with cells. It is contemplated that the incubated cells do not exhibit any, or are substantially free of, incubator induced edge effects.

19. The method of embodiment 18, wherein the concentrator mask is removed from the multiwell plate prior to placing the multiwell plate into the incubator.

20. The method of embodiment 17, further comprising analyzing the cells in the multiwell plate to obtain an optical measurement.

### Example 1

Cells were seeded, expanded and analyzed in multiwell plates of the present technology that comprise a continuous ring. These cells were compared to control cells that were seeded, expanded and analyzed in multiwell plates comprising standard wells comprising no continuous ring. The tested continuous ring had a wall height of 0.2 mm and an inner diameter of 2.0 mm. The same number of cells (4,500) were analyzed for both well types.

After the cells were seeded and expanded, oxygen consumption rates (OCR) were measured using an Agilent XFp instrument. The rates were measured by obtaining an optical measurement. The results demonstrated that the same number of cells (4,500) produced three times higher signal intensity when cultured in the wells comprising a continuous ring as compared to the cells cultured in standard wells that comprise no continuous ring.

In view of this disclosure it is noted that the methods and apparatus can be implemented in keeping with the present teachings. Further, the various components, materials, structures and parameters are included by way of illustration and example only and not in any limiting sense. In view of this disclosure, the present teachings can be implemented in other applications and components, materials, structures and equipment to implement these applications can be determined, while remaining within the scope of the appended claims.

We claim:

1. A multiwell plate for a cell population in a liquid medium, the multiwell plate comprising:
a frame having a frame surface and frame sides extending from the frame surface;
a plurality of wells, each well having an open end, a closed end opposite the open end, and at least one wall between the open end and the closed end; wherein the open end of each of the wells is surrounded by the frame surface; wherein the closed end comprises a well surface between and contacting the at least one wall; and
at least one continuous ring on the well surface of the closed end of one or more of the wells.

2. The multiwell plate of claim 1, wherein the at least one continuous ring is configured to define at least one cell seeding area on the well surface.

3. The multiwell plate of claim 1, wherein the multiwell plate comprises more than one continuous ring configured to define more than one cell seeding area.

4. The multiwell plate of claim 1, wherein the at least one continuous ring has a height of about 0.01 mm to about 2 mm.

5. The multiwell plate of claim 1, wherein the at least one continuous ring has an inner diameter of about 0.5 mm to about 6.0 mm.

6. A concentrator mask for seeding cells in liquid medium into a multiwell plate, the concentrator mask comprising:
a frame having a frame surface and frame sides extending from the frame surface;
at least one funnel extending from the frame surface, wherein the at least one funnel comprises a first open end and a second open end;
wherein the first open end is connected to the frame surface and has a larger diameter than the second open end.

7. The concentrator mask of claim 6, wherein the second open end comprises a distal elastomer portion.

8. The concentrator mask of claim 6, wherein the concentrator mask comprises at least 8 funnels.

9. The concentrator mask of claim 6, wherein the funnels have an inner diameter of about 0.5 mm to about 6.0 mm.

10. A method of seeding cells in a central portion of a culture well, the method comprising:
adding a liquid medium comprising cells into the at least one cell seeding area on the well surface of claim 2.

11. The method of claim 10, further comprising incubating the culture well after seeding with cells.

12. The method of claim 11, wherein the incubated cells are substantially free of incubator induced edge effects.

13. A cell seeding system comprising a multiwell plate and a concentrator mask:
wherein the multiwell plate comprises a frame having a frame surface and frame sides extending from the frame surface; a plurality of wells, each well having an open end, a closed end opposite the open end, and at least one wall between the open end and the closed end; wherein the open end of each of the wells is surrounded by the frame surface; wherein the closed end comprises a well surface between and contacting the wall; and at least one continuous ring on the well surface of the closed end of each well;
wherein the concentrator mask comprises a frame having a frame surface and frame sides extending from the frame surface; at least one funnel extending from the frame surface, wherein the at least one funnel has a first open end and a second open end; and wherein the first open end is connected to the frame surface and has a larger diameter than the second open end; and
wherein the at least one funnel is configured to fit into at least one of the plurality of wells so that the second open end of the funnel form an interface with the continuous ring on the well surface of the closed end of one or more of the wells.

14. The system of claim 13, wherein the interface between the second open end and the continuous ring is a gap sufficient to reduce liquid diffusion.

15. The system of claim 13, wherein the second open end comprises a distal elastomer portion.

16. The system of claim 15, wherein the interface between the distal elastomer portion of the second open end and the continuous ring forms a liquid-tight seal.

17. A method of seeding cells in a multiwell plate, the method comprising;
adding a liquid medium comprising cells into the first open end of the cell seeding system of claim 13, wherein the cells are deposited into an area circumscribed by the at least one continuous ring on the well surface.

18. The method of claim 17, wherein the multiwell plate is moved to an incubator after seeding with cells.

19. The method of claim 18, wherein the concentrator mask is removed from the multiwell plate prior to moving the multiwell plate into the incubator.

20. The method of claim 17, further comprising analyzing the cells in the multiwell plate to obtain an optical measurement.
